(19) United States
(12) Patent Application Publication
(10) Pub. No.: US 2024/0358775 A1
(43) Pub. Date: Oct. 31, 2024

(54) METHOD OF ALTERING MICROBIOMES TO PRODUCE BENEFICIAL EFFECTS

(71) Applicant: CARDIAI TECHNOLOGIES, Calgary (CA)

(72) Inventors: Minal Borkar Tripathi, Calgary (CA); Sidharth Singh Bhinder, Calgary (CA); Anmol Singh Kapoor, Calgary (CA); Amrita Kaur, Calgary (CA)

(21) Appl. No.: 18/646,939

(22) Filed: Apr. 26, 2024

Related U.S. Application Data
(60) Provisional application No. 63/498,611, filed on Apr. 27, 2023.

(51) Int. Cl. A61K 35/741; A61K 36/06

(52) U.S. Cl. CPC A61K 35/741; A61K 36/06

(57) ABSTRACT

The present disclosure relates to a method for modulating a microbiome of a human subject to promote health and prevent disease comprising steps of identifying one or more bacterial strains associated with health from the isolated strains and administering the identified bacterial strains to a subject in need thereof to alter and modulate the microbiome and promote growth of beneficial microbes, inhibit growth of harmful microbes, or modulate an immune system.

### CROSS-REFERENCE TO RELATED APPLICATIONS

This application claims the benefit of U.S. Provisional Application No. 63/498,611 titled “Method of Altering Microbiomes to Produce Beneficial Effects” filed by the applicant on, Apr. 27, 2023, which is incorporated herein by reference in its entirety.

### FIELD OF THE INVENTION

Embodiments of the present invention relate to the field of microbiology and more particularly, relate to methods of altering microbiomes using microbiome modulation to promote health and prevent disease.

### DESCRIPTION OF THE RELATED ART

A microbiome can include an ecological community of commensal, symbiotic, and pathogenic microorganisms that are associated with an organism. Characterization of the human microbiome is a complex process. The human microbiome includes over 10 times more microbial cells than human cells, but characterization of the human microbiome is still in nascent stages such as due to limitations in sample processing techniques, genetic analysis techniques, and resources for processing large amounts of data.

A typical healthy individual's body is inhabited with trillions of microbes across various body sites called microbiomes. Some examples of microbiome sites include skin, intestinal, stomach, gut, oral, conjunctival, and vaginal. To better understand the role of these microbiomes and how they affect physiology and disease state we can analyze what microbes comprise a microbiome and how they correlate or affect the health status and clinical response of an individual. For example, the human gut microbiome is known to play a key role in many health conditions, including obesity, gastrointestinal health, nutrient absorption, and drug metabolism among others.

Present knowledge has clearly established the role of microbiome associations with multiple health conditions, and has become an increasingly appreciated mediator of host genetic and environmental factors on human disease development.

The microbiome is suspected to play at least a partial role in a number of health/disease-related states (e.g., preparation for childbirth, diabetes, auto-immune disorders, gastrointestinal disorders, rheumatoid disorders, neurological disorders, etc.). Further, the microbiome plays an essential role in human health, with dysbiosis of the microbiome linked to a range of diseases, including inflammatory bowel disease, diabetes, and obesity. Furthermore, the microbiome may mediate effects of environmental factors on human, plant, and/or animal health. Given the profound implications of the microbiome in affecting a subject's health, efforts related to the characterization of the microbiome, the generation of insights from the characterization, and the generation of therapeutics configured to rectify states of dysbiosis should be pursued.

The most commonly used methods for microbiome modulation involve antibiotics. However, these methods are limited in their effectiveness and often result in unwanted side effects.

Further, the current methods and systems for analyzing the microbiomes of humans and/or providing therapeutic measures based on gained insights have, however, left many questions unanswered. Thus, there is a need in the field of microbiology for a new and useful method and/or system for modulating the microbiome to promote health and/or prevent diseases, microorganism-related health conditions and/or the associated relationships (e.g., specific features associated with microorganisms and/or conditions, etc.), such as for individualized and/or population-wide use. Therefore, the present invention provides alternative methods of microbiome modulation.

### SUMMARY OF THE INVENTION

Embodiments of the present invention relate to a method for modulating a microbiome of a subject to promote health and prevent disease comprising steps of identifying one or more bacterial strains associated with health from the isolated strains and administering the identified bacterial strains to a subject in need thereof to modulate the microbiome and promote health. In particular, the microbiome of the subject is altered to promote growth of beneficial microbes, inhibit growth of harmful microbes, or modulate an immune system. The subject is a human.

In accordance with an embodiment of the present invention, the bacterial strains associated with health are identified by:
a. obtaining a sample from a healthy subject;
b. isolating the bacteria from the sample; and
c. identifying the bacterial strains present in the sample.

In accordance with an embodiment of the present invention, the bacterial strains are administered to the subject in the form of a probiotic. The probiotic is administered orally, intranasally, or via any other suitable route of administration.

In accordance with another embodiment of the present invention, the bacterial strains are administered to the subject in the form of a pharmaceutical composition. The pharmaceutical composition is formulated for oral, nasal, or topical administration.

In accordance with an embodiment of the present invention, the probiotic comprises at least one species of bacteria or yeast and at least one type of dietary fiber.

In accordance with an embodiment of the present invention, the microbiome alteration is performed for the prevention or treatment of a disease or disorder selected from the group consisting of obesity, diabetes, inflammatory bowel disease, irritable bowel syndrome, and allergies.

Another embodiment of the present invention relates to a microbial agent for use in altering the microbiome of a subject, comprising at least one probiotic, prebiotic, postbiotic, or a combination thereof.

In accordance with an embodiment of the present invention, the postbiotic comprises at least one metabolite produced by microbial fermentation.

Yet another embodiment of the present invention, the composition for altering the microbiome of a subject, comprising an effective amount of at least one microbial agent selected from the group consisting of probiotics, prebiotics, postbiotics, and combinations thereof, and a pharmaceutically acceptable carrier.

The carrier is selected from the group consisting of tablets, capsules, powders, granules, and liquids.

The composition as claimed in claim 16, wherein the probiotic comprises at least one species of bacteria or yeast.

The foregoing objectives of the present invention are attained by providing an

### BRIEF DESCRIPTION OF THE DRAWINGS

So that the manner in which the above-recited features of the present invention is understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

The invention herein will be better understood from the following description with reference to the drawings, in which:

FIG. 1 is a flowchart illustrating a method for modulating a microbiome of a subject in accordance with one embodiment of the present invention;

FIG. 2 is a method for identifying the bacterial strains associated with health in accordance with an embodiment of the present invention.

It should be noted that the accompanying figure is intended to present illustrations of exemplary embodiments of the present disclosure. This figure is not intended to limit the scope of the present disclosure. It should also be noted that the accompanying figure is not necessarily drawn to scale.

### DETAILED DESCRIPTION OF THE INVENTION

In the following detailed description, numerous specific details are set forth in order to provide a thorough understanding of the embodiment of the invention as illustrative or exemplary embodiments of the invention, specific embodiments in which the invention may be practised are described in sufficient detail to enable those skilled in the art to practice the disclosed embodiments. However, it will be obvious to a person skilled in the art that the embodiments of the invention may be practised with or without these specific details. In other instances, well-known methods, procedures and components have not been described in detail so as not to unnecessarily obscure aspects of the embodiments of the invention.

The following detailed description is, therefore, not to be taken in a limiting sense, and the scope of the present invention is defined by the appended claims and equivalents thereof. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. References within the specification to “one embodiment,” “an embodiment,” “embodiments,” or “one or more embodiments” are intended to indicate that a particular feature, structure, or characteristic described in connection with the embodiment is included in at least one embodiment of the present invention.

Although the terms first, second, etc. may be used herein to describe various elements, these elements should not be limited by these terms. These terms are generally only used to distinguish one element from another and do not denote any order, ranking, quantity, or importance, but rather are used to distinguish one element from another. Further, the terms “a” and “an” herein do not denote a limitation of quantity, but rather denote the presence of at least one of the referenced items.

The conditional language used herein, such as, among others, “can,” “may,” “might,” “may,” “e.g.,” and the like, unless specifically stated otherwise, or otherwise understood within the context as used, is generally intended to convey that certain embodiments include, while other embodiments do not include, certain features, elements and/or steps.

Disjunctive language such as the phrase “at least one of X, Y, Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

### The Following Brief Definition of Terms Shall Apply Throughout the Present Invention:

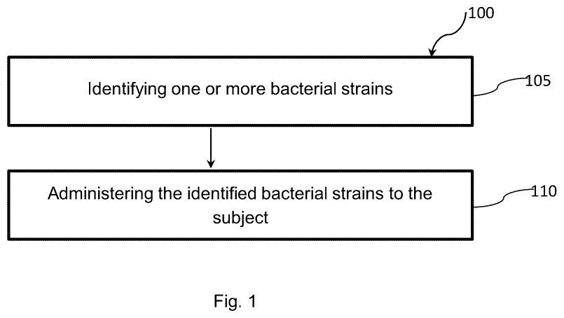

FIG. 1 is a flowchart illustrating a method for modulating a microbiome of a subject in accordance with one embodiment of the present invention. The method starts at step 105.

At step 105, one or more bacterial strains associated with health are identified from the isolated strains.

At step 110, the identified bacterial strains are administered to a subject in need thereof to modulate the microbiome and promote health. In particular, the subject is a human subject.

In accordance with an embodiment of the present invention, the bacterial strains are administered to the subject in the form of a probiotic. The probiotic is administered orally, intranasally, or via any other suitable route of administration.

In embodiments of the foregoing aspects, the bacterial stains are bacterial populations comprising one or more bacterial species consisting but not limited to, lactic acid bacteria, bifido bacteria, enterococci, bacilli, and combinations thereof.

In another embodiment of the foregoing aspect, the bacterial population may be an anti-inflammatory bacterial population.

In accordance with an embodiment of the present invention, the bacterial strains may be selected based on their ability to produce a desired effect in the microbiome, such as promoting a healthy gut flora, improving digestion, reducing inflammation, and/or promoting a healthy immune system. \In accordance with an embodiment of the present invention, the probiotic is a slow-release probiotic formulated to slowly release the one or more microbial strains over a period of time. For example, the probiotic formulation in a capsule form may be designed to release the microbial strains gradually in the gastrointestinal tract, promoting prolonged colonization and modulation of the microbiome.

[Alternatively, the probiotic may be a fast release probiotic formulated to release the one or more microbial strains over a short period of time.

In an exemplary example, the probiotic nasal spray formulation containing selected bacterial strains, such as lactic acid bacteria and bacilli, aimed at modulating the nasal and respiratory microbiome. This formulation is designed to promote respiratory health, reduce the risk of infections, and alleviate symptoms of allergic rhinitis.

In another exemplary example, rectal suppository probiotic formulation comprising microbial strains specifically chosen for their ability to colonize the lower gastrointestinal tract effectively. This formulation is intended for individuals with gastrointestinal issues such as inflammatory bowel disease or irritable bowel syndrome, aiming to restore balance to the gut microbiome and alleviate symptoms.

For oral administration the formulation can be formulated as but not limited to a capsule, tablet, liquid, powder, or any other suitable form.

In an exemplary example, the probiotic capsule contains a single bacterial species that are selected for its efficacy in treating a specific condition, such as Lactobacillus acidophilus for vaginal health or Bifidobacterium infantis for irritable bowel syndrome.

In another exemplary example, the probiotic formulation formulated for oral administration comprises the bacterial strains present in an amount effective to alter the microbiome of the human subject to whom the probiotic composition is orally administered. The probiotic formulation may be administered to the human subject, in one or more doses, as an oral nutritional supplement.

In accordance with alternate embodiment of the present invention, the bacterial strains are administered to the subject in the form of a pharmaceutical composition. The pharmaceutical composition is formulated for oral, nasal, or topical administration.

In another exemplary example, the pharmaceutical composition may be administered rectally and/or vaginally.

In accordance with another embodiment of the present invention, the probiotic may be administered orally, topically, or through any other routes of administration known or to be developed in future.

In an exemplary example, a topical probiotic cream may contain microbial strains selected for their ability to promote skin health and combat conditions such as acne, eczema, or psoriasis. This formulation delivers live probiotic bacteria directly to the skin, where they can modulate the local microbiome and reduce inflammation.

In accordance with an embodiment of the present invention, the probiotic comprises at least one species of bacteria or yeast and at least one type of dietary fiber.

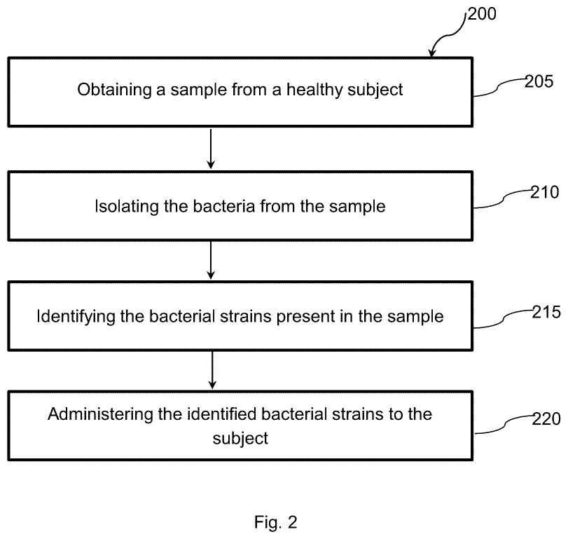

FIG. 2 is a method for identifying the bacterial strains associated with health in accordance with an embodiment of the present invention.

At step 205, a sample is obtained from a healthy subject;

At step 210, the bacteria is isolated from the sample

At step 215, the bacterial strains present in the sample are identified.

At step 220, the identified bacterial strains are administered to a subject in need thereof to modulate the microbiome and promote health. In particular, the subject is a human subject.

In accordance with an embodiment of the present invention, the microbiome alteration is performed for the prevention or treatment of a disease or disorder selected from the group consisting of obesity, diabetes, inflammatory bowel disease, irritable bowel syndrome, and allergies.

In accordance with an embodiment of the present invention, a microbial agent for used in altering the microbiome of a subject, comprising at least one probiotic, prebiotic, postbiotic, or a combination thereof. The postbiotic in the microbial comprises at least one metabolite produced by microbial fermentation. Further, the probiotic comprises at least one species of bacteria or yeast and at least one type of dietary fiber.

In accordance with an embodiment of the present invention, a composition for altering the microbiome of a subject, comprising an effective amount of at least one microbial agent selected from the group consisting of probiotics, prebiotics, postbiotics, and combinations thereof, and a pharmaceutically acceptable carrier.

The carrier is selected from the group consisting of tablets, capsules, powders, granules, and liquids.

The probiotic comprises at least one species of bacteria or yeast and at least one type of dietary fiber.

The postbiotic comprises at least one metabolite produced by microbial fermentation.

The compositions can be administered by any route suitable for the delivery of disclosed compositions for treating, inhibiting, or preventing a dysbiosis, or diseases and disorders associated with a dysbiosis, promoting a healthy gut flora, improving digestion, reducing inflammation, and/or promoting a healthy immune system including, but are not limited to orally, sublingually, rectally, parentally (e.g., intravenous injection (i.v.), intracranial injection (i.e.); intramuscular injection (i.m.), intraperitoneal injection (i.p.), and subcutaneous injection (s.c.) and intraosseous infusion (i.o.)), transdermally (using any standard patch), extracorporeally, inhalation, topically or the like, including topical intranasal administration or administration by inhalant. The compositions and dosage forms described herein can be administered by e.g., intradermal, ophthalmic, (intra) nasally, local, non-oral, such as aerosol, inhalation, subcutaneous, intramuscular, buccal, sublingual, (trans) rectal, vaginal, intra-arterial, and intrathecal, transmucosal (e.g., sublingual, lingual, (trans) buccal, (trans) urethral, vaginal (e.g., trans- and perivaginally), intravesical, intrapulmonary, intraduodenal, intragastrical, intrabronchial, etc. In preferred embodiments, the pharmaceutical compositions and dosage forms described herein are administered by routes selected from oral, topical, (trans) dermal, (intra) nasal, and rectal. In certain embodiments, the (intra) nasal administration is achieved via aerosol or inhalation.

In one embodiment of the foregoing aspect, administration of the probiotic reduces inflammation in the gastrointestinal tract of the human subject having an autoimmune or inflammatory disorder such as but not limited to graft-versus-host disease (GVHD), an inflammatory bowel disease (IBD), ulcerative colitis, Crohn's disease, multiple sclerosis (MS), systemic lupus erythematosus (SLE), type I diabetes, rheumatoid arthritis, Sjögren's syndrome, and Celiac disease.

In another embodiment of the foregoing aspect, the human subject administered with probiotic may have dysbiosis. The dysbiosis may be a gastrointestinal dysbiosis or a distal dysbiosis.

In some embodiments of the foregoing aspect, the administration of the formulation at a first site reduces inflammation at a distal site such as blood, skin, vagina, liver, spleen, fallopian tubes, uterus, or a combination thereof in the human subject.

In an embodiment of the foregoing aspect, the formulation can also be formulated as a food product.

In another aspect, the food product may be a medical food product or a non medical food product.

In some embodiments of the foregoing aspects, the food product is an infant formula.

In some embodiments of the foregoing aspects, the food product is a yogurt. In some embodiments of the foregoing aspects, the food product is a beverage, e.g., chilled beverage.

In some embodiments of the foregoing aspects, the composition is administered daily through the consumption of a food product comprising the pharmaceutical product

The probiotic formulation may also comprise one or more other components, such as prebiotics, vitamins, minerals, and/or other beneficial compounds. Prebiotic is an ingredient that allows specific changes, both in the composition and/or activity in the gastrointestinal microbiota that may (or may not) confer benefits upon the host. In some embodiments, a prebiotic can be a comestible food or beverage or ingredient thereof. In some embodiments, a prebiotic may be a selectively fermented ingredient. Prebiotics may include complex carbohydrates, amino acids, peptides, minerals, or other essential nutritional components for the survival of the bacterial composition. Prebiotics include, to, amino but are not limited acids, biotin, fructooligosaccharide, galactooligosaccharides, hemicelluloses (e.g., arabinoxylan, xylan, xyloglucan, and glucomannan), inulin, chitin, lactulose, mannan oligosaccharides, oligofructose-enriched inulin, gums (e.g., guar gum, gum arabic and carrageenan), oligofructose, oligodextrose, tagatose, resistant maltodextrins (e.g., resistant starch), trans-galactooligosaccharide, pectins (e.g., xylogalactouronan, citrus pectin, apple pectin, and rhamnogalacturonan-I), dietary fibers (e.g., soy fiber, sugar beet fiber, pea fiber, corn bran, and oat fiber) and xylooligosaccharide.

In embodiments of the foregoing aspects, the probiotic comprises live bacterial species. The live bacterial species comprise live microbes, microbes that are lyophilized, freeze-dried, and/or substantially dehydrated, or the composition may comprise bacterial or fungal spores or virion.

In another embodiment, the probiotic may comprise non-live bacterial species, such as bacterial lysates or bacterial-derived products. These formulations offer stability and convenience, particularly in situations where live probiotic formulations may not be feasible or practical, such as in shelf-stable supplements or processed foods.

In one embodiment, the probiotic comprises a single bacterial species. In alternative embodiments the probiotic formulation comprises a combination of bacterial species. Each bacterial species may be purified from a fecal material obtained from a single human subject, or from two or more human subjects.

In an embodiment of the foregoing aspects, the bacterial species are selected based on their ability to treat or prevent a particular condition associated with dysbiosis of the microbiome, selected from the group consisting of obesity, diabetes, inflammatory bowel disease, and mental health disorders.

In another embodiment of the foregoing aspects, the bacterial species are selected based on their ability to colonize the subject's microbiome and restore balance to the microbiome. Further, the bacterial species can be selected based on their ability to produce metabolites that promote health, their ability to compete with harmful bacteria, or their ability to modulate immune function.

In yet another embodiment, the bacterial species are selected based on their ability to treat or prevent a particular condition associated with dysbiosis of the microbiome. For example, the composition can be formulated to treat or prevent conditions such as obesity, diabetes, inflammatory bowel disease, and mental health disorders.

In accordance with alternative embodiment of the present invention, the formulation may include at least one microbiome-modulating agent selected from prebiotics, postbiotics, synbiotics, or combinations thereof, a carrier suitable for delivery of the microbiome-modulating agent to the subject. The microbiome-modulating agent is selected from the group consisting of dietary fibers, oligosaccharides, polyphenols, lactic acid bacteria, bifidobacteria, butyrate-producing bacteria, bacteriophages, and bacterial metabolites. Further, the microbiome-modulating agent is present in an effective amount to modulate the microbiome of the subject.

In a case that no conflict occurs, the embodiments in the present disclosure and the features in the embodiments may be mutually combined. The foregoing descriptions are merely specific implementations of the present disclosure, but are not intended to limit the protection scope of the present disclosure. Any variation or replacement readily figured out by a person skilled in the art within the technical scope disclosed in the present disclosure shall fall within the protection scope of the present disclosure. Therefore, the protection scope of the present disclosure shall be subject to the protection scope of the claims.

The foregoing descriptions of specific embodiments of the present technology have been presented for purposes of illustration and description. They are not intended to be exhaustive or to limit the present technology to the precise forms disclosed, and obviously many modifications and variations are possible in light of the above teaching. The embodiments were chosen and described in order to best explain the principles of the present technology and its practical application, to thereby enable others skilled in the art to best utilize the present technology and various embodiments with various modifications as are suited to the particular use contemplated. It is understood that various omissions and substitutions of equivalents are contemplated as circumstance may suggest or render expedient, but such are intended to cover the application or implementation without departing from the spirit or scope of the claims of the present technology.

What is claimed for:

1. A method for modulating a microbiome of a subject to promote health and prevent disease, comprising:
a. identifying one or more bacterial strains associated with health from the isolated strains; and
b. administering the identified bacterial strains to a subject in need thereof to modulate the microbiome and promote health;
wherein the microbiome of the subject is altered to promote growth of beneficial microbes, inhibit growth of harmful microbes, or modulate an immune system.

2. The method as claimed in claim 1, wherein the bacterial strains associated with health are identified by:
a. obtaining a sample from a healthy subject;
b. isolating the bacteria from the sample; and
c. identifying the bacterial strains present in the sample
d. administering the identified bacterial strains to a subject in need thereof to modulate the microbiome and promote health.

3. The method as claimed in claim 1, wherein the bacterial strains are administered to the subject in the form of a probiotic.

4. The method as claimed in claim 1, wherein the probiotic is administered orally, intranasally, or via any other suitable route of administration.

5. The method as claimed in claim 1, wherein the bacterial strains are administered to the subject in the form of a pharmaceutical composition.

6. The method as claimed in claim 1, wherein the pharmaceutical composition is formulated for oral, nasal, or topical administration.

7. The method as claimed in claim 1, wherein the probiotic comprises at least one species of bacteria or yeast and at least one type of dietary fiber.

8. The method as claimed in claim 1, wherein the subject is a human.

9. The method as claimed in claim 1, wherein the microbiome alteration is performed for the prevention or treatment of a disease or disorder selected from the group consisting of obesity, diabetes, inflammatory bowel disease, irritable bowel syndrome, and allergies.

10. The microbial agent of claim 8, wherein a postbiotic comprises at least one metabolite produced by microbial fermentation.

11. A microbial agent for use in altering the microbiome of a subject, comprising at least one probiotic, prebiotic, postbiotic, or a combination thereof.

12. The microbial agent as claimed in claim 11, wherein the postbiotic comprises at least one metabolite produced by microbial fermentation.

13. The microbial agent as claimed in claim 11, wherein the probiotic comprises at least one species of bacteria or yeast.

14. The microbial agent as claimed in claim 11, wherein the prebiotic comprises at least one type of dietary fiber.

15. The microbial agent as claimed in claim 11, wherein the subject is a human.

16. A composition for altering the microbiome of a subject, comprising an effective amount of at least one microbial agent selected from the group consisting of probiotics, prebiotics, postbiotics, and combinations thereof, and a pharmaceutically acceptable carrier.

17. The composition as claimed in claim 16, wherein the carrier is selected from the group consisting of tablets, capsules, powders, granules, and liquids.

18. The composition as claimed in claim 16, wherein the probiotic comprises at least one species of bacteria or yeast.

19. The composition as claimed in claim 16, wherein the prebiotic comprises at least one type of dietary fiber.

20. The composition as claimed in claim 16, wherein the postbiotic comprises at least one metabolite produced by microbial fermentation.
